(19) United States
(12) Patent Application Publication
(10) Pub. No.: US 2009/0043126 A1
(43) Pub. Date: Feb. 12, 2009

(54) Process and Methods for the Preparation of Gabapentin and Its Intermediates

(76) Inventors: Jordi Bosch I Llado, Girona (ES); Carmen Arnalot Aguilar, Girona (ES); Maria Carmen Burgarolas Montero, Santa Maria De Palautordera (ES)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 11/885,122

(22) PCT Filed: Feb. 28, 2005
(86) PCT No.: PCT/IB2005/002496

§ 371 Date: Aug. 4, 2008

(51) Int. Cl. C07C 227/16

(52) U.S. Cl. 562/507

(57) ABSTRACT

The invention provides a process for preparing gabapentin and its intermediates. The process generally involves treating gabapentin lactam with an aqueous solution of hydrobromic acid to yield gabapentin hydrobromide salt as an intermediate, which can be isolated by filtration. The gabapentin hydrobromide salt can then be hydrolyzed with a base, such as an organic amine, to yield gabapentin that can be isolated by filtration.

### BACKGROUND OF THE INVENTION

1. Field of the Invention

The invention relates to a process for preparing gabapentin and its intermediates. In particular, the invention relates to a multi-step process for converting gabapentin lactam (2-azaspiro[4,5]decan-3-one or 1-aminomethyl-1-cyclohexane-acetic acid lactam) to gabapentin utilizing hydrobromic acid (“HBr”).

2. Discussion of the Related Art

Gabapentin (1-aminomethyl-1-cyclohexane-acetic acid) is an anticonvulsant having the chemical structure:

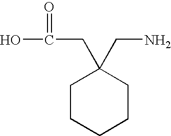

Anticonvulsants are used to control seizure disorders. In this regard, gabapentin has been successfully used to treat and/or control seizures associated with cerebral diseases including, for example, epilepsy. Gabapentin has also been used to manage postherpetic neuralgia (i.e., the pain after “shingles”) and it may also be useful for mood stabilization and treating anxiety. Although gabapentin is related to the brain chemical gamma aminobutyric acid (GABA), its exact mechanism of action remains unknown.

A number of conventional methods are known for preparing gabapentin. Some of these methodologies are summarized in PCT/IN2003/000246 (Publication No. WO 2004/101489), which is incorporated herein by reference in its entirety. Such methods include, for example, preparing gabapentin from cyclohexyl-1,1-diacetic acid via formation of the gabapentin hydrochloride (“gabapentin HCl”) salt. Gabapentin HCl is then converted to gabapentin by treating with a basic ion exchanger followed by crystallization from a solvent such as ethanol/ether.

Other synthetic methods for producing gabapentin from the gabapentin HCl salt intermediate include converting the hydrochloride salt into the free amino acid by (1) pouring a deionized water solution of the salt over an ion exchange column, (2) eluting with deionized water, (3) producing a slurry from the eluate, (4) adding an alcohol to the slurry, (5) centrifuging and (6) drying the slurry to obtain the free amino acid. Similarly, anhydrous gabapentin has been prepared from gabapentin HCl by treating the hydrochloride salt with a basic ion exchange resin and then either concentrating or spray drying the solution to obtain anhydrous gabapentin. In another methodology, the hydrochloride salt was treated with an ion exchange resin and the aqueous solution obtained was concentrated and any remaining water was removed by azeotropic distillation. The wet gabapentin was then diluted with isopropyl alcohol and cooled to yield anhydrous gabapentin.

Gabapentin has also been prepared by dissolving gabapentin HCl in isopropyl alcohol and treating with activated carbon. The suspension was then heated under predetermined parameters and washed with isopropyl alcohol. Thereafter, tributylamine was added and the resulting gabapentin was isolated by filtration and washed with methanol.

The foregoing synthetic methods, however, often require using large amounts of an ion exchanger for lengthy periods of time to lower the level of chloride ions to desirable and/or acceptable levels.

Other methods are also known for producing gabapentin that do not involve using the hydrochloride salt intermediate. Such methods, for example, utilize a cyanic intermediate which is hydrogenated under severe conditions to produce the free amino acid.

Gabapentin is known to exist in at least three polymorphic forms that differ from each other based primarily on their crystal structure and associated water content. Form I is the monohydrate (gabapentin hydrate). Gabapentin hydrate form I can be considered a “pseudopolymorphic” form of anhydrous gabapentin and generally exists as large crystals having undefined shapes. Form II is anhydrous gabapentin, which exists as plate shaped crystals. Form II is frequently referred to as pharmaceutical grade or “commercial” gabapentin. Form III is another form of anhydrous gabapentin, which usually exists as small rhomboidal crystals. The different forms of gabapentin can be readily distinguished based upon their IR spectra and X-ray diffraction patterns as discussed in U.S. Pat. No. 6,255,526 and PCT Application No. PCT/US97/23164 (Publication No. WO 98/28255), which are incorporated herein by reference in their entirety.

At least one method for producing gabapentin form II has been developed in which gabapentin hydrochloride is reacted with an amine in order to precipitate gabapentin form III from solution. This method generally involves pre-treating gabapentin HCl with a second solvent to remove inorganic salts, then dissolving it in a first solvent and subsequently treating with an amine to form a precipitate. The resulting anhydrous gabapentin had physical characteristics differing from the gabapentin usually used in pharmaceutical preparations. The anhydrous polymorph obtained by this process was designated as form III and could be crystallized with methanol to yield gabapentin form II. Similarly, a process has been reported for isolating gabapentin form II starting from gabapentin hydrochloride in which the chloride ion was eliminated by precipitating it as an insoluble salt and thus releasing gabapentin in aqueous solution as the free amino acid.

U.S. Pat. No. 6,521,787, which is incorporated herein by reference in its entirety, discloses a method for obtaining gabapentin by spray drying and/or turbo-drying an aqueous solution of gabapentin to yield one polymorphic form of gabapentin from which pharmaceutical grade gabapentin is obtained through crystallization from various solvents.

### SUMMARY OF THE INVENTION

The present invention relates to a process of preparing gabapentin and its intermediates. More specifically, the invention relates to a process of hydrolyzing gabapentin lactam (1-aminomethyl-1-cyclohexane-acetic acid lactam) (A) with aqueous HBr to form gabapentin hydrobromide (“gabapentin HBr”) (B) followed by treatment with an organic amine to yield gabapentin (C).

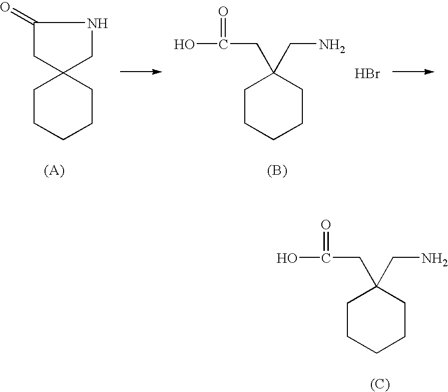

Modifications and variations of the present invention are possible and envisioned in light of the above descriptions. It is therefore to be understood that within the scope of the attached detailed description, examples and claims, the invention may be practiced otherwise than as specifically described.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates a flow chart of a multistep process for preparing gabapentin and its intermediates;

FIG. 2 illustrates the IR spectrum of gabapentin HBr produced by the disclosed method and process;

FIG. 3 illustrates the ¹H NMR spectrum of gabapentin HBr produced by the disclosed method and process;

FIG. 4 illustrates the ¹³C NMR spectrum gabapentin HBr produced by the disclosed method and process;

FIG. 5 illustrates the X-Ray diffraction pattern of gabapentin HBr produced by the disclosed method and process; and

FIG. 6 illustrates the X-Ray diffraction pattern of gabapentin produced by the disclosed method and process.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition and as will be appreciated by one of skill in the art, the invention may be embodied as a method, system or process.

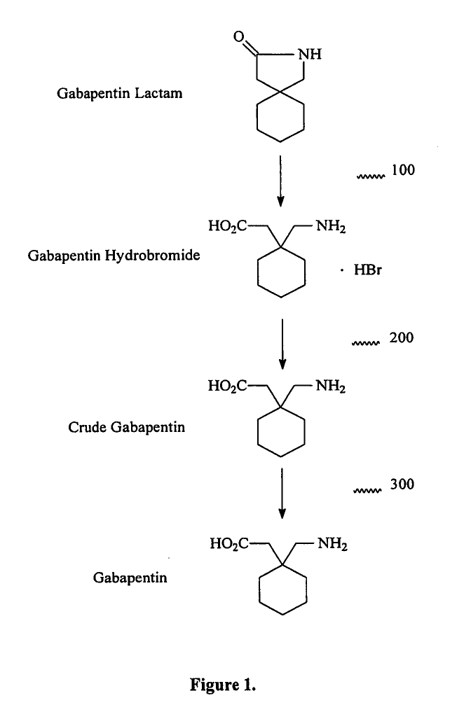

FIG. 1 illustrates a flow chart of a multistep process for preparing gabapentin and its intermediates. The process begins at step 100 where gabapentin lactam is hydrolyzed with an aqueous hydrobromic acid solution to yield gabapentin HBr. The aqueous HBr is generally prepared by mixing 1 volume of water with 2 volumes of HBr (e.g., 1 L H₂O and 2 L HBr). In one embodiment, one kilogram of gabapentin lactam is combined with every three liters of the aqueous HBr solution (i.e., gabapentin lactam:water:HBr=1 kg:1 L:2 L). In another embodiment, the aqueous HBr solution is approximately 6N. After the gabapentin lactam and aqueous HBr are combined, the solution is heated to reflux temperature and stirred at that temperature for at least three hours. After the solution is cooled and the resulting solid isolated, the filtrate can be optionally subjected to an additional cycle of reflux and stirring to yield another crop of isolatable product. The resulting solid gabapentin HBr is generally obtained by filtration and/or centrifugation and is then washed with an organic solvent (e.g., methylethylketone and/or isopropyl alcohol). Preferably, the organic solvent is a ketonic solvent such as methylethylketone.

Step 100 is characterized by requiring a shortened reaction times. Additionally, step 100 utilizes less solvent materials throughout the synthesis and work-up (as well as re-utilizing the filtrate materials for additional crop yields), thus resulting in less solvent waste materials.

In step 200, the isolated gabapentin HBr from step 100 is neutralized by the addition of a base, preferably an organic amine, such as tributylamine or diethylamine. Other suitable amines include triethylamine, diisopropylamine, diisopropylethylamine, dibutylamine, di-(2-ethylhexylamine), dicyclohexylamine.

The organic amine or other base (e.g., NaOH) is added in a quantity sufficient to produce a pH value preferably between approximately 7.2±0.8 (i.e., the approximate isoelectric point of gabapentin±0.8), although higher pH values are also suitable under various conditions. The organic amine or other base is generally added to the gabapentin HBr, which has been dissolved in an aqueous organic solvent (e.g., methanol and/or a 5% water/acetone solution), at an elevated temperature (e.g., between 30° and 50° C.). Alternatively, the organic amine or other base can be added at room temperature, and other suitable solvents include methanol/water/isopropyl alcohol mixtures, ethanol, isopropyl alcohol, methanol, methylethylketone and combinations thereof and combinations further including water. Optionally, the solution of gabapentin hydrobromide can be filtered in order to eliminate any insoluble materials prior to the addition of the organic amine or other base.

Alternatively, the gabapentin HBr may also be neutralized using an ion exchange resin as disclosed in U.S. Pat. No. 6,528,682, which is incorporated herein by reference in its entirety.

In step 300, the crude gabapentin obtained in step 200 is further purified by recrystallization. In step 300, approximately 1 part of “crude gabapentin” is suspended in 2.5 volumes of methanol and heated to reflux. Water is added to the solution until the crude gabapentin dissolves (approximately 0.67 volumes of H₂O). Other suitable solvents include methanol/water/isopropyl alcohol mixtures as well as mixtures further including or replacing isopropyl alcohol with methylethylketone and/or acetone. Optionally, the solution can be filtered to remove any insoluble material(s) after the crude gabapentin is dissolved in solution. The solution is then cooled to ambient temperature to initiate precipitation. Optionally, precipitation can also be initiated by seeding with gabapentin. The resulting solid gabapentin is then washed with isopropyl alcohol (approximately 2.5 volumes) and dried under vacuum.

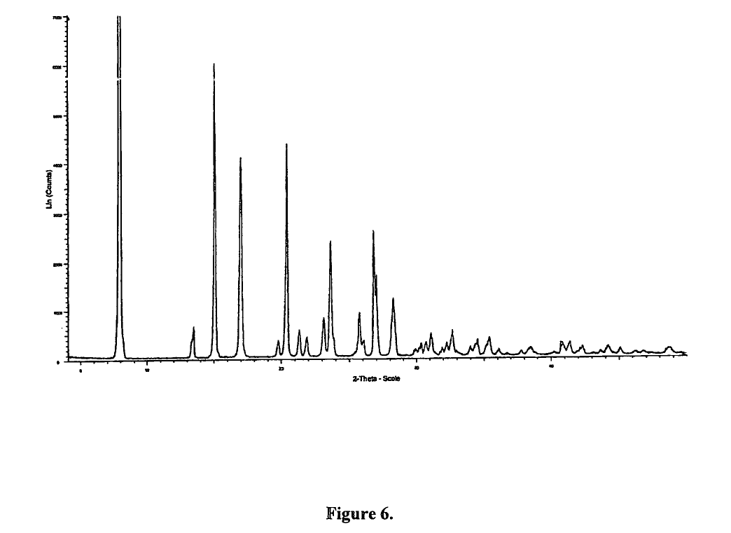

The resulting gabapentin has a crystal structure (see FIG. 6) corresponding to that of pharmaceutical grade gabapentin (sold under the trade name Neurontin®), which has been referred to as gabapentin form II and is the pharmaceutical standard for gabapentin.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

### SPECIFIC EXAMPLES

The following examples are for illustrative purposes only and are not intended, nor should they be interpreted to, limit the scope of the invention.

General Experimental Conditions: Loss on drying measurements were conducted at 40° C. under vacuum until a constant weight was obtained. HPLC analysis was performed according to the method described in Pharmacopoeia Forum Vol. 28(2) [March-April 2002] (under the heading “Gabapentin Capsules”).

### Example 1

Step 1: A 15 liter reactor was purged with nitrogen and charged with 1.0 kg of gabapentin lactam (2-azaspiro[4,5]decan-3-one or 1-aminomethyl-1-cyclohexane-acetic acid lactam) (6.527 mol, 1.0 molar equivalent), 2.130 kg of water (18.13 molar equivalents) and 6.60 kg of hydrobromic acid 48% aqueous solution (4.40 L of HBr 48%, 3.168 kg of HBr 100%, 39.16 mol, 6.0 molar equivalents). The resulting colorless, transparent solution was heated to ˜100-108° C. over 4 hours and stirred for 6 hours at reflux temperature. The solution was then cooled to ˜0-5° C. over an 8 hour period and stirred at that temperature for 6 hours. The solution was then filtered to produce 1.07 kg of wet gabapentin HBr, which corresponded to 0.94 kg of dry material.

The same reactor was next charged with the filtrate (7.97 kg) from the previous step. The filtrate solution was heated to reflux (˜108° C.) over 4 hours and stirred for 6 hours at reflux temperature. The solution was then cooled to ˜40±2° C. and seeded with 1 g of gabapentin HBr from the previous step. Thereafter, the solution was further cooled to ˜0-5° C. over 5 hours and stirred at this temperature for 8 hours. The solution was then filtered and dried under suction to produce an additional 0.30 kg of moist gabapentin HBr.

The first and second yields of gabapentin HBr were combined and charged into the same reactor with 0.944 kg of isopropyl alcohol (1.21 L). The suspension was cooled and maintained at ˜0-5° C. for 2 hours and then filtered. The resulting solid was washed with 0.079 kg isopropyl alcohol to yield 1.334 kg of moist gabapentin HBr (loss on drying 25.05%; yield: 60.74%).

Step 2: A 2 L reactor equipped with a thermometer, reflux condenser and mechanical stirrer was purged with nitrogen and charged with 370 g of moist gabapentin HBr and 370 mL of methanol (˜1.33 volumes relative to gabapentin HBr). The suspension was heated to ˜40-45° C. and filtered to remove any residual inorganic material. The resulting solution was neutralized with 120.7 mL of diethylamine (˜1.05 molar equivalents relative to gabapentin HBr), added at ˜35-40° C. over 50 minutes. Additional diethylamine was added to adjust the pH to between 7.5 and 8.0. The suspension was then stirred for 10 minutes, the pH was checked and the mixture heated to reflux (˜66° C.). Water was added (˜41 mL) to the suspension until dissolution was complete (˜0.15 volumes relative to gabapentin HBr). The solution was cooled to ˜30° C. and 471 mL of isopropyl alcohol (˜1.7 volumes) was added over 10-15 min. The suspension was then cooled to ˜0-5° C. over 25 minutes, stirred for 2 hours and filtered. The resulting solid was washed twice with 30 mL of cold isopropyl alcohol and dried under suction to give moist gabapentin (134.1 g, loss on drying 2%; partial yield: 69.8%; total yield from gabapentin lactam: 42.40%).

Step 3: A 2 L reactor equipped with a thermometer, reflux condenser and magnetic stirrer was purged with nitrogen and charged with 130 g of moist gabapentin and 318 mL of methanol (˜2.5 volumes relative to gabapentin). The suspension was heated to reflux (˜66° C.) and water was added until dissolution was complete (˜85 mL, ˜0.67 volumes relative to gabapentin). The solution was cooled to ˜20° C. over 40 minutes and 320 mL of isopropyl alcohol was added (˜2.5 volumes relative to gabapentin). The suspension was cooled to ˜0-5° C. over 25 minutes, stirred for 2 hours at this temperature and filtered. The resulting white crystalline solid was washed twice with 25 mL of cold isopropyl alcohol and dried under vacuum at ˜40° C. to yield gabapentin (110.6 g; partial yield: 87.00%; total yield from gabapentin lactam: 36.88%)

Analytical data: Assay (HClO₄): 100.98%; Description: white crystalline powder; Water content (KF method): 0.10%; Loss on drying: 0.12%; pH (2% solution in water): 7.39; Bromide content: 23 ppm; Residue on ignition: 0.05%; Heavy metals: <20 ppm; HPLC (% area): 99.4%.

### Example 2

Step 1: A 2 L reactor was purged with nitrogen and charged with 150 g of gabapentin lactam (0.979 mol, 1.0 molar equivalent), 164.3 g of water and 500.1 g of hydrobromic acid 48.31% aqueous solution (333.4 mL of HBr 48.31%, 241.6 g of HBr 100%, 2.986 mol, 3.05 molar equivalents). The resulting transparent, slightly yellow solution was heated to ˜108-114° C. and stirred for 6 hours at reflux temperature. The solution was then cooled to ˜0-5° C. and stirred at that temperature for 6 hours. The solution was then filtered to yield 217.9 g of wet gabapentin HBr.

The same reactor was then charged with the filtrate from the previous step, heated to reflux (˜108-114° C.) and stirred for 6 hours at reflux temperature. The solution was then cooled to ˜0-5° C. and stirred at this temperature for 9 hours. After filtration, the product was dried under suction to give an additional 52.5 g of moist gabapentin HBr.

The first and second yields of the gabapentin HBr were combined (266.7 g of wet gabapentin HBr, which corresponded to 200.7 g of the dry material) and charged into the same reactor with 270 g of methylethylketone (336 mL). The suspension was cooled and maintained at ˜0-5° C. for 2 hours and then filtered. The resulting solid was washed with 27 g of methylethylketone to yield 178.3 g of moist gabapentin HBr (loss on drying 5.53%; yield: 69.20%).

Step 2: A 2 L reactor equipped with a thermometer, reflux condenser and mechanical stirrer was purged with nitrogen and charged with 106.7 g of moist gabapentin HBr (corresponding to 100 g of dry gabapentin HBr) and 600 mL of a 5% water/acetone mixture (˜6 volumes relative to gabapentin HBr). The suspension was neutralized with 98.4 mL of tributylamine (˜1.04 mol relative to gabapentin HBr) added at room temperature over 50 minutes. Additional tributylamine was added to adjust the pH to between 7.4 and 8.0. The suspension was then stirred for 1.5 hours, the pH checked and the mixture filtered. The resulting white solid was washed twice with 30 mL of the 5% water/acetone mixture and dried under vacuum to yield moist gabapentin (66.56 g; loss on drying 3.11%; partial yield: 93.5%; total yield from gabapentin lactam: 64.70%; Bromide content: 3000 ppm).

Step 3: A 1 L reactor equipped with a thermometer, reflux condenser and mechanical stirrer was purged with nitrogen and charged with 56.77 g of moist gabapentin and 137.5 mL of methanol (2.5 volumes relative to gabapentin). The suspension was heated to reflux (˜66° C.) and water was added until dissolution was complete (36.8 mL, ˜0.67 volumes relative to gabapentin). The solution was cooled to room temperature over 40 minutes and 137.5 mL of isopropyl alcohol was added (˜2.5 volumes relative to gabapentin). The suspension was cooled to ˜0-5° C. over 25 minutes, stirred for 2 hours at this temperature and filtered. The resulting white, crystalline solid was washed twice with 15 mL of cold isopropyl alcohol and dried under vacuum at ˜40° C. to yield gabapentin (46.7 g, partial yield: 84.93%, total yield from gabapentin lactam: 54.95%).

Analytical data: Assay: 100.11%; Description: white crystalline powder; Water content (KF method): 0.06%; pH (in a 2% water solution): 7.38; Residue on ignition: 0.00%; Heavy metals: <20 ppm; Bromide content: 11 ppm; HPLC (% area): 99.382%; gabapentin lactam is not detected; any other unknown impurity is present in a content less than a 0.05%.

### Example 3

Step 1: A 1 L reactor was charged with 75 g of gabapentin lactam (0.4895 mol, 1.0 molar equivalent), 82.15 mL of water and 250.0 g of hydrobromic acid 48.31% aqueous solution (166.7 mL of HBr 48.31%, 120.79 g of HBr 100%, 1.493 mol, 3.05 molar equivalents). The resulting transparent, slightly yellow solution was heated to reflux (˜108-114° C.) and stirred for 6 hours at reflux temperature. The solution was cooled to ˜0-5° C. and stirred at that temperature for 6 hours. The solution was then filtered to yield 107.7 g of wet gabapentin HBr.

The filtrate was analyzed to determine the gabapentin lactam and HBr content. The same reactor was then charged with 75% of the filtrate and sufficient quantities of gabapentin lactam, HBr and water (72.0 g of gabapentin lactam, 123.0 g of aqueous HBr 48.31%, 25 mL of H₂O, respectively) to achieve the initial reaction conditions. The solution was heated to reflux (˜108-114° C.) and stirred for 6 hours. The solution was then cooled to ˜0-5° C. and stirred at this temperature for 6 hours. After filtration, the product was dried under suction to yield 135.2 g of moist gabapentin HBr.

The filtrate was then again analyzed for gabapentin lactam and HBr content. Thereafter, 75% of the filtrate was charged to the reactor with 67.4 g of gabapentin lactam, 140.0 g of HBr 48.31% and 24.6 g of water. The solution was heated to reflux (˜108-114° C.) and stirred for 6 hours. The solution was then cooled to 0-5° C. and stirred at that temperature for 5 hours. After filtration, the product was dried under suction to give 122.0 g of moist gabapentin HBr.

Next, 75% of the filtrate from the previous step was charged to the reactor with 64.1 g of gabapentin lactam, 19.5 mL of water and 132 g hydrobromic acid 48.31% aqueous solution to give the initial reaction conditions. The mixture was heated to reflux for 6 hours, cooled and stirred at ˜0-5° C. for 6 hours. After filtration, 127.8 g of moist gabapentin HBr was obtained.

A 2 L reactor was charged with the four (4) combined crops of gabapentin HBr (485.0 g of moist gabapentin HBr, which corresponded to 351.1 g of dry material) and 610 mL of methylethylketone. The suspension was cooled and maintained at ˜0-5° C. for 2 hours and filtered. The resulting solid was washed with 50 mL of methylethylketone to yield 320.3 g of moist gabapentin HBr (loss on drying 9.29%; yield: 64.3%).

Step 2: A 2 L reactor was charged with 110.2 g of moist gabapentin HBr (corresponding to 100 g of dry gabapentin HBr) and 600 mL of a 5% water/acetone mixture (˜6 volumes relative to gabapentin HBr). The suspension was neutralized by addition of 98.4 mL of tributylamine (˜1.04 relative to gabapentin HBr) at room temperature over 50 minutes. Additional tributylamine was added to adjust the pH to between 7.4 and 8. The suspension was then stirred for 1.5 hours, the pH checked and the mixture filtered. The resulting white solid was washed twice with 30 mL of a 5% water/acetone mixture and dried under suction to give moist gabapentin (65.33 g, loss on drying 3.73%, partial yield: 92.60%, total yield from gabapentin lactam: 59.52%).

Step 3: A 1 L reactor equipped with a thermometer, reflux condenser and mechanical stirrer was purged with nitrogen and charged with 64.5 g of moist gabapentin and 155.3 mL of methanol (˜2.5 volumes relative to gabapentin). The suspension was heated to reflux (˜66° C.) and water was added until dissolution was complete (41.6 mL, ˜0.67 volumes relative to gabapentin). The solution was cooled to room temperature over 40 minutes and 155.3 mL of isopropyl alcohol was added (˜2.5 volumes relative to gabapentin). The suspension was cooled to ˜0-5° C. over 25 min, stirred for 2 hours at this temperature and filtered. The resulting white crystalline solid was washed twice with 15 mL of cold isopropyl alcohol and dried under vacuum at ˜40° C. to yield gabapentin (49.98 g, partial yield: 80.5%; global yield from gabapentin lactam: 47.89%).

Analytical data: bromide content: 49 ppm; HPLC (% area): 99.4%.

### Example 4

### Gabapentin HBr Analytical Sample

Step 1: A 2 L reactor was purged with nitrogen and charged with 200 g of gabapentin lactam (1.305 mol, 1.0 molar equivalent), 438 g of water and 1293 g of hydrobromic acid 49.02% aqueous solution (862 mL of HBr 49.02%, 633.68 g of HBr 100%, 7.832 mol, 6 molar equivalents). The resulting transparent, slightly yellow solution was heated to ˜108-114° C. and stirred for 6 hours at reflux temperature. The solution was then cooled to ˜0-5° C. and stirred for 15-16 hours at this temperature. The solution was then filtered to yield 251.3 g of wet gabapentin HBr.

The filtrate from the previous step was charged to the same reactor and then heated and stirred at reflux (˜108-114° C.) and for 6 hours. The solution was then cooled to between ˜10-20° C. and stirred at this temperature for 15 hours. Precipitation began following seeding with a small portion of the gabapentin HBr obtained in the previous step. After stirring 3 hours at this temperature, the product was filtered and dried by suction to yield 69.7 g of moist gabapentin HBr.

The first and second yields were then combined (321 g) in the same reactor with 450.87 g of acetone (570 mL). The suspension was cooled and maintained at ˜0-5° C. for 1.5 hours. The resulting solid was filtered and washed with 79.1 g of acetone (100 mL) to yield 238.24 g of moist gabapentin HBr (loss on drying 9.9%, 214.7 g of dried product, yield: 65.24%).

An analytical sample of gabapentin hydrobromide was obtained by suspending the obtained solid twice in isopropyl alcohol. The solid obtained was dried at ˜40° C. under vacuum until a constant weight was obtained.

Analytical data: Description: white crystalline solid; Assay (HClO₄): 99.58%; Water content (KF method): 3.21%; Melting point: 116.4-116.9° C.; Elemental analysis: C₉H₁₇NO₂.HBr.0.5H₂O (261.16 g/mol): C 41.30%, H 7.40%, N 5.34%, Br 30.90%

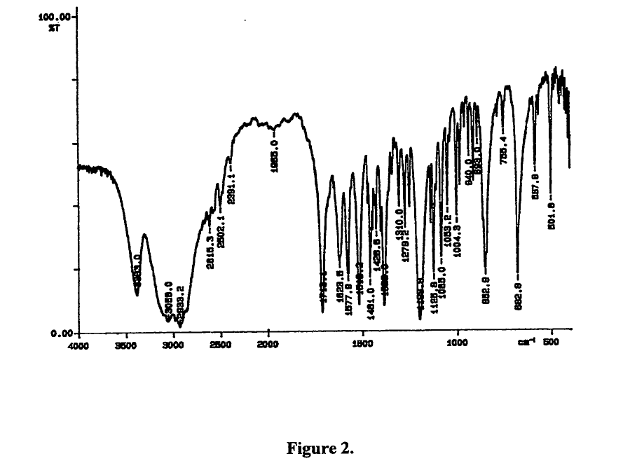

FIG. 2 illustrates the IR spectrum of the gabapentin HBr from Example 4 and Table 1 (below) identifies the IR peaks of the gabapentin HBr.

TABLE 1
IR (KBr) Peaks of Gabapentin HBr₁

| Frequency, ν (cm⁻¹) | Assignment |
|---|---|
| 3600-2300 | O—H |
| (Maxima at 3383, 3058, 2933, 2615, 2502) | C—H (stretch) |
| | NH₃⁺(stretch) |
| 1713 | C═O (stretch) |

Notes:
¹Other Absorption Bands: 1624, 1578, 1519, 1461, 1447, 1437, 1429, 1405, 1388, 1310, 1279, 1253, 1199, 1141, 1126, 1085, 1053, 1004, 986, 853 and 683 cm⁻¹.

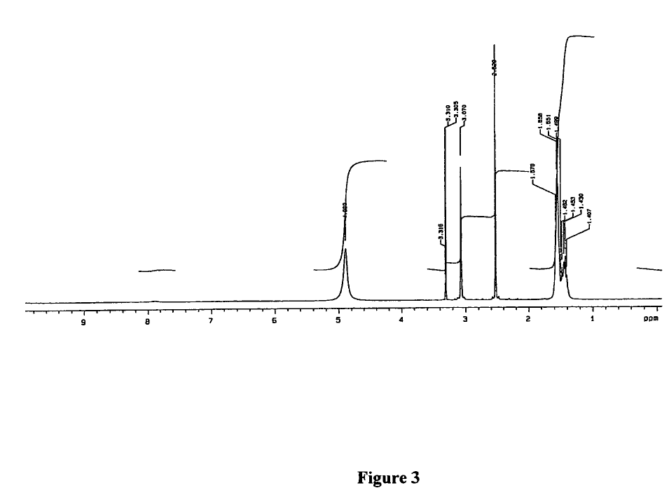

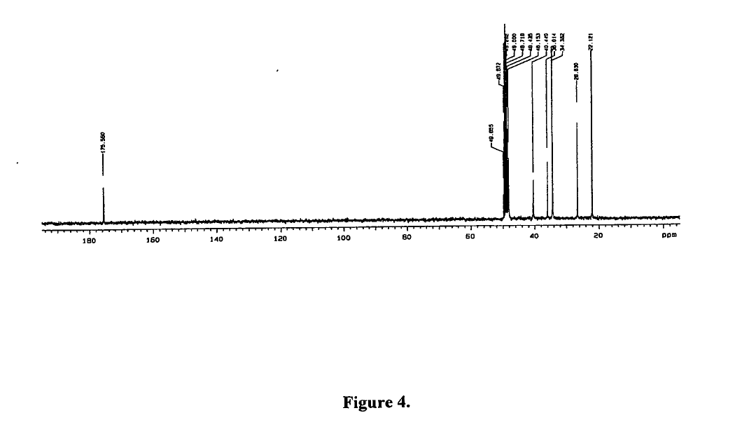

FIGS. 3 and 4 illustrate the ¹H and ¹³C NMR spectra of the gabapentin HBr from Example 4 and Tables 2 and 3 (below) identify the chemical shifts and peak assignments of the gabapentin HBr.

TABLE 2
¹H NMR of Gabapentin HBr (300 MHz, CD, OD)¹

| Chemical Shift | | Number of H | |
|---|---|---|---|
| δ (ppm) | Multiplicity | Atoms | Assignment |
| 1.35-1.64 | compl. signal | 10 | Cyclohexyl H |
| 2.53 | s | 2 | CH₂CO₂H |
| 3.07 | s | 2 | CH₂NH₃⁺ |
| 4.89 | s (broad) | 4 | Mobile H |
| 7.90 | s (broad) | 1 | CH₂CO₂H |

Notes:
¹The signal at δ 3.31 ppm corresponds to CD₃OD.

TABLE 3
¹³C NMR of Gabapentin HBr (75.4 MHz, CD, OD)¹

| Chemical Shift | Kind of | |
|---|---|---|
| δ (ppm) | Carbon Atom | Assignment |
| 22.1 | CH₂ | Cyclohexane C3 (5) |
| 26.6 | CH₂ | Cyclohexane C4 |
| 34.4 | CH₂ | Cyclohexane C2 (6) |
| 36.0 | C | Cyclohexane C1 |
| 40.4 | CH₂ | CH₂CO₂⁻ |
| 48.2 | CH₂ | CH₂NH₃⁺ |
| 175.6 | C | CH₂CO₂⁻ |

Notes:
¹The signal at δ 49.0 ppm corresponds to solvent.

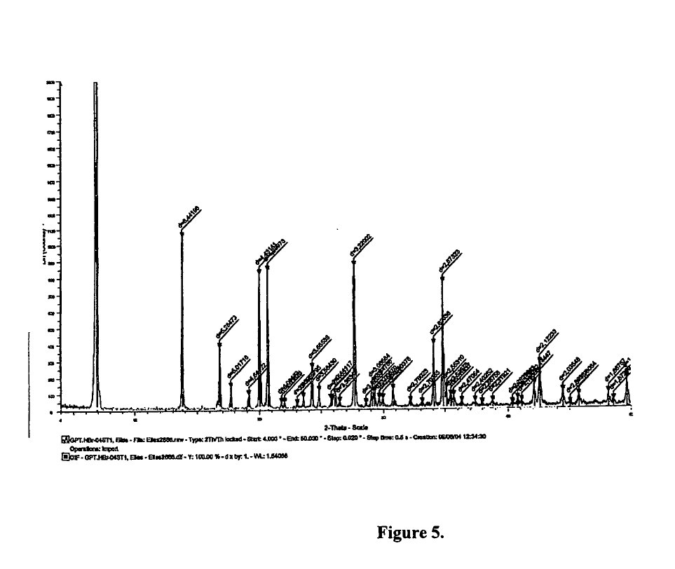

FIG. 5 demonstrates the X-Ray powder diffraction pattern of the gabapentin HBr from Example 4 and Table 4 identifies the main diffraction peaks of the gabapentin HBr.

TABLE 4
X-Ray Diffraction Peaks of Gabapentin HBr^{1,2}

| d (A) | 2-Theta | I (Cnts) |
|---|---|---|
| 12.88604 | 6.844 | 8959 |
| 6.44196 | 13.735 | 1061 |
| 5.28473 | 16.762 | 385 |
| 5.01716 | 17.663 | 145 |
| 4.43141 | 20.020 | 824 |
| 4.28973 | 20.689 | 845 |
| 3.66588 | 24.259 | 259 |
| 3.58430 | 24.820 | 120 |
| 3.41117 | 26.101 | 102 |
| 3.22002 | 27.681 | 869 |
| 3.06584 | 29.103 | 145 |
| 3.04766 | 29.280 | 110 |
| 2.90378 | 30.766 | 124 |
| 2.63038 | 34.056 | 394 |
| 2.57323 | 34.836 | 764 |
| 2.55310 | 35.120 | 130 |
| 2.14447 | 42.101 | 147 |
| 2.12233 | 42.562 | 276 |
| 2.03548 | 44.472 | 106 |
| 1.83398 | 49.670 | 179 |

Notes:
¹Intensities below 100 are not tabulated.
²Obtained using a SIEMENS D5000 X-ray powder diffractometer equipped with a vertical goniometer, graphite secondary monochromator and scintillation detector; Radiation source = Cu anode tube; Sample holder = glass; Scanning speed = 1 second per step with steps of 0.02 deg.

### Example 5

Step 1: A 500 mL, 3-necked round bottom flask was purged with nitrogen and charged with 50 g of gabapentin lactam (0.326 mol, 1.0 molar equivalent), 54.75 g of water and 166.7 g of hydrobromic acid 48.31% aqueous solution (111.1 mL of HBr 48.31%, 80.53 g of HBr 100%, 0.995 mol, 3.05 molar equivalent). The resulting transparent, slightly yellow solution was heated to reflux (˜108-114° C.) and stirred for 3 hours at reflux temperature. The solution was then cooled to ˜0-5° C. and stirred at that temperature overnight. The solution was then filtered to yield 83.02 g of wet gabapentin HBr.

The reactor was charged with the filtrate from the previous step and heated to reflux (˜108-114° C.) and stirred at that temperature for 3 hours. The solution was then cooled to ˜10-15° C. and seeded with 0.2 g of gabapentin HBr from the previous step. Thereafter, the solution was further cooled to ˜0-5° C. and stirred at this temperature overnight. After filtration, the product was dried under suction to yield 21.74 g of moist gabapentin HBr.

The same reactor was then charged with the wet gabapentin HBr from the first and second crops (corresponding to 62.61 g of dry material) and 96.72 g of methylethylketone (120 mL). The suspension was cooled and maintained at ˜0-5° C. for approximately 2 hours. The solution was then filtered and washed twice with 8.06 g of methylethylketone (10 mL) to yield 47.48 g of moist gabapentin HBr (loss on drying 6.73%, yield: 59.27%).

Step 2: A 500 mL 3-necked round bottom flask was charged with 45.88 g of moist gabapentin HBr (corresponding to 42.79 g of dry gabapentin HBr) and 256.74 mL of a 5% water/acetone mixture (˜6 volumes relative to gabapentin HBr). The suspension was heated to 40-46° C. and produced an almost clear solution. The solution was then cooled to ˜30° C. and neutralized by the addition of tributylamine. After initially adding approximately 2.1 mL (i.e., ˜5%) of a total of 42.53 mL of tributylamine (33.05 g, 0.1783 mol, ˜1.05 molar equivalents), the solution was seeded with 0.2 g of gabapentin to initiate precipitation. Thereafter, the remaining tributylamine was added at room temperature over 50 minutes and the suspension was stirred for 1.5 hours (pH ˜6.28). Additional tributylamine was added (4.5 mL, 3.5 g, 0.019 mol; 1.16 total molar equivalents) to adjust the pH to between 7.4 and 8.0. The suspension was then stirred for 15 minutes, the pH checked and the mixture filtered. The resulting white solid was washed twice with 10 mL of a 5% water/acetone mixture and dried under suction to yield moist gabapentin (27.88 g, loss on drying 0.29%; partial yield: 95.66%; total yield from gabapentin lactam: 56.70%; bromide content: 0.85%).

Step 3: A 250 mL 3-necked round bottom flask equipped with a thermometer, reflux condenser and magnetic stirrer was purged with nitrogen and charged with 25.65 g of moist gabapentin (corresponding to 25.58 g of dry material) and 64 mL of methanol (50.69 g, ˜2.5 volumes relative to gabapentin). The suspension was heated to reflux (˜66° C.) and water was added until dissolution was complete (20.5 mL, ˜0.8 volumes relative to gabapentin). The solution was cooled to room temperature over 40 minutes and 64 mL of isopropyl alcohol (50.24 g, ˜2.5 volumes relative to gabapentin) was added. The suspension was then further cooled to ˜0-5° C. over 25 minutes, stirred for 2 hours and filtered. The resulting white crystalline solid was washed twice with 7 mL of cold isopropyl alcohol and dried under vacuum at ˜40° C. to yield gabapentin (20.48 g; partial yield: 80.06%; total yield from gabapentin lactam: 45.39%).

Analytical data: Bromide content: 14 ppm; HPLC (% area): 99.374%, gabapentin lactam not detected.

### Example 6

Step 1: 274.0 kg of water and 828 kg of concentrated hydrobromic acid (48%) were mixed together. To the solution, 250 kg of gabapentin lactam was added with stirring. The mixture was then boiled under reflux (˜110° C.) for 3 hours. The reaction mixture was cooled to ˜0-5° C. over 6-8 hours and stirred at this temperature for at least an additional 6 hours. The resulting precipitate was isolated by filtration.

The filtrate was recovered and boiled under reflux (˜110° C.) for 3 hours. The reaction mixture was then cooled to ˜40° C. over 8 hours. The cooled filtrate can optionally be seeded with gabapentin hydrobromide. The filtrate was further cooled to ˜0-5° C. over 5 hours and stirred at this temperature for a minimum of 8 additional hours. The resulting precipitate was obtained by filtration.

The two crops of gabapentin HBr were combined and stirred with 457 kg of methylethylketone for at least 2 hours at ˜0-5° C. The resulting wet gabapentin HBr was isolated by filtration (yield: 63.2%).

Step 2: The wet gabapentin HBr obtained in step 1 was combined with 1283 kg of acetone and 85 kg of water and the temperature was adjusted to between ˜35-40° C. over approximately 1 hour. Thereafter, 22 kg of tributylamine was added over 15 minutes. The solution was next seeded with gabapentin and the suspension was cooled to ˜20-25° C. over approximately 1 hour. Additional tributylamine was added to adjust the pH to between 7.5 and 8. The mixture was then stirred and maintained at ˜20-25° C. for 1 hour and the pH was adjusted as necessary. The resulting wet (crude) gabapentin was isolated by filtration (partial yield: 94.1%).

Step 3: The wet crude gabapentin obtained in step 2 was combined with 358 kg of methanol and heated to reflux (˜66° C.). Water was added until dissolution (a minimum of 130 kg) was complete. The mixture was then cooled to ˜60° C. and filtered to remove any particulates which were washed with methanol. The solution was then further cooled to ˜20-25° C. Following the addition of 362 kg of isopropyl alcohol, the mixture was cooled to ˜0-5° C. and stirred at this temperature for a minimum of 2 hours. The resulting crystalline gabapentin was isolated by filtration and dried (partial yield: 77.0%).

FIG. 6 illustrates the X-Ray diffraction pattern of the gabapentin produced in Example 6.

### Example 7

Step 1: A 2 liter reactor was purged with nitrogen and charged with 297.7 g of gabapentin lactam (1.943 mol, 1.0 molar equivalent), 437 g of water and 1310 g of hydrobromic acid 48% aqueous solution (873 mL of HBr 48%, 628.8 g of HBr 100%, 7.77 mol, 4 molar equivalents). The resulting transparent, slightly yellow solution was heated to ˜108-114° C. and stirred for 6 hours at reflux temperature. The solution was then cooled to ˜0-5° C., stirred for 6 hours and filtered to yield 444.07 g of wet gabapentin HBr.

The same reactor was charged with the filtrate from the previous step, heated to reflux (˜108-114° C.) and stirred for 6 hours at this temperature. The solution was then cooled to ˜0-5° C. and stirred at this temperature overnight. The resulting product was isolated by filtration and suction dried to yield 96.02 g of moist gabapentin HBr.

The same reactor was charged with the filtrate from the previous step, heated to reflux (˜108-114° C.) and stirred for 6 hours at this temperature. The solution was then cooled to ˜0-5° C. and stirred at this temperature for two days. The resulting product was isolated by filtration and suction dried to yield 26.36 g of moist gabapentin HBr.

A 1 L reactor was purged with nitrogen and charged with 558.7 g of the combined crops of gabapentin HBr (corresponding to 424.7 g of dry material) and 394.07 g of isopropyl alcohol (˜502 mL). The suspension was cooled and maintained at 0-5° C. for 2 hours and filtered. The resulting solid was washed twice with 15.7 g of isopropyl alcohol to yield 392.5 g of moist gabapentin HBr (loss on drying 17.1%, yield: 76.6%).

Step 2: 87.74 g of moist gabapentin HBr (corresponding to ˜72.7 g of dry material) obtained in Step 1 was dissolved in 590.0 g (˜745 mL) of methanol. The resulting solution was passed through an ion exchange column two times. The obtained fractions were collected and reduced to 125 mL. The resulting suspension was heated to reflux (˜66° C.) and water was added until dissolution was complete (29 mL, ˜0.59 volumes relative to gabapentin). The solution was then cooled to room temperature over 40 minutes and 125 mL of isopropyl alcohol was added (˜2.5 volumes relative to gabapentin). The suspension was further cooled to ˜0-5° C. over 25 minutes, stirred for 2 hours and then filtered. The resulting white, crystalline solid was washed twice with 12 mL of cold isopropyl alcohol and dried under vacuum at ˜40° C. to yield gabapentin (31.97 g, partial yield: 64.7%, total yield from gabapentin lactam: 49.56%, bromide content: 24.9 ppm). The product was not recrystallized.

### Example 8

### Synthesis of Gabapentin HBr

Step 1: A 250 mL, 3-necked round bottom flask was purged with nitrogen and charged with 20 g of gabapentin lactam (0.1305 mol, 1.0 molar equivalent), 28.3 g of water and 88.01 g of hydrobromic acid 48% aqueous solution (58.7 mL of HBr 48%, 42.25 g of HBr 100%, 0.522 mol, 4 molar equivalents). The resulting transparent, slightly yellow solution was heated to ˜108-114° C. and stirred for 6 hours at reflux temperature. The solution was then cooled to 0-5° C., stirred for 8 hours and filtered to yield 22.9 g of wet gabapentin HBr.

The same reactor was charged with the filtrate from the previous step, heated to reflux (˜108-114° C.) and stirred for 6 hours at this temperature. The solution was then cooled to ˜40° C. and seeded with a small quantity of the solid obtained in the previous step. The seeded solution was then cooled to ˜0-5° C. and maintained at this temperature overnight. The resulting precipitate was isolated by filtration and dried by suction to yield 6.56 g of moist gabapentin HBr.

A 250 mL, 3-necked round bottom flask was purged with nitrogen and charged with 28.52 g of the combined crops of gabapentin HBr (corresponding to 23.68 g of dry material) and 27.94 g of acetone (35.32 mL). The suspension was cooled and maintained at ˜0-5° C. for 2 hours and filtered. The resulting solid was washed twice with 4 g of acetone to yield 21.19 g of moist gabapentin HBr (loss on drying 7.50%, yield: 62.06%).

Tables 5-8 summarize the results of the foregoing examples.

TABLE 5
Summary of Step 1

| | | | Lactam/ | | | |
|---|---|---|---|---|---|---|
| | Lactam/H₂O/HBr | H₂O/HBr | Volume | Reflux | | Yield³ |
| Ex. | (kg/L/L) | (L/L) | (kg/L) | Time¹ | Work-up² | (%) |
| 1 | 1/2.13/4.4 | 1/2.0657 | 1/6.53 | 6 h + | Centrifugation | 60.7 |
| | | | | ML 6 h | Suspension in | |
| | | | | (2 crops) | IPA | |
| 2 | 1/1.0953/2.2227 | 1/2.0293 | 1/3.3293 | 6 h + | Centrifugation | 69.2 |
| | | | | ML 6 h | Suspension in | |
| | | | | (2 crops) | MEK | |
| 3 | 1/1.0953/2.2227 | 1/2.0425 | 1/3.318 | 6 h + | Centrifugation | 64.3 |
| | | | | MLR 6 h + | Suspension in | |
| | | | | MLR 6 h + | MEK | |
| | | | | MLR 6 h | | |
| | | | | (4 crops) | | |
| 4 | 1/2.19/4.31 | 1/1.968 | 1/6.5 | 6 h + | Centrifugation | 65.2 |
| | | | | ML 6 h | Suspension in | |
| | | | | (2 crops) | ACETONE | |
| 5 | 1/1.095/2.2376 | 1/2.22 | 1/3.3317 | 3 h + | Centrifugation | 59.3 |
| | | | | ML 3 h | Suspension in | |
| | | | | (2 crops) | MEK | |
| 6 | 1/1.096/2.2228 | 1/2.0281 | 1/3.3188 | 3 h + | Centrifugation | 63.2 |
| | | | | ML 3 h | Suspension in | |
| | | | | (2 crops) | MEK | |
| 7 | 1/1.5888/2.9324 | 1/1.8557 | 1/4.5212 | 6 h + | Centrifugation | 67.3 |
| | | | | ML 6 h + | Suspension in | |
| | | | | ML 6 h | IPA | |
| | | | | (3 crops) | | |
| 8 | 1/1.415/2.935 | 1/2.0742 | 1/4.35 | 6 h + | Centrifugation | 62.1 |
| | | | | ML 6 h | Suspension in | |
| | | | | (2 crops) | ACETONE | |

Notes:
¹ML = Filtrate subjected to additional reaction cycle; MLR = Filtrate adjusted to initial reaction conditions and submitted to new reaction cycle.
²IPA = isopropyl alcohol; MEK = methylethylketone.
³Based on loss on drying data.

TABLE 6
Summary of Step 2 Results

| | Gabapentin | | | |
|---|---|---|---|---|
| | HBr | | | Yield³ |
| Ex. | Solvent¹ | Solvent/Temperature | Amine² | (%) |
| 1 | IPA | MeOH | DIE | 69.8 |
| | | 40-45° C. (solution) | | |
| 2 | MEK | 5% water/acetone | TBA | 93.5 |
| | | (suspension) | | |
| 3 | MEK | 5% water/acetone | TBA | 92.6 |
| 5 | MEK | 5% water/acetone | TBA | 95.6 |
| | | 40-46° C. (solution) | | |
| 6 | MEK | Water/acetone | TBA | 94.1 |
| | | 35-40° C. (solution) | | |
| 7 | IPA | MeOH | N/A | 64.7 |

Notes:
¹IPA = isopropyl alcohol; MEK = methylethylketone; MeOH = methanol.
²DIE = diethylamine; TBA = tributylamine.
³Based on loss on drying data.

Table 7 illustrates various neutralization pH values obtained using different amines and solvent conditions to neutralize gabapentin HBr.

TABLE 7
Neutralization pH Values With Different Amines and Solvents

| | | pH | Step 2 |
|---|---|---|---|
| Amine | Solvent | Value | Yield¹ (%) |
| Diethylamine | MeOH/H₂O/IPA | 8.52 | 97.4% |
| | 2.22/1/3.77 | | |
| | MeOH/H₂O/IPA | 9.37 | 73.0% |
| | 19/1/24.28 | | |
| | MeOH/H₂O/IPA | 8.37 | 70.1% |
| | 10/1/12.78 | | |
| | MeOH/H₂O/IPA | 8.61 | 70.0% |
| | 13.3/1/17 | | |
| | MeOH/H₂O/IPA | 8.11 | 77.0% |
| | 8.86/1/11.33 | | |
| | MeOH/H₂O/IPA | 8.90 | 66.5% |
| Diisopropylethylamine | 2.22/1/3.77 | 8.08 | 76.4% |
| Diethylamine | MeOH/H₂O/IPA | 8.5 | 63.8% |
| | 7.82/1/10 | | |
| | 5% H₂O/IPA | 8.62 | 67.9% |
| | MeOH/H₂O/IPA | 8.5 | 67.5% |
| | 4.43/1/5.66 | | |
| | 10% H₂O/IPA | 8.21 | 75.4% |
| | 5% H₂O/2% MeOH/MEK | 7.00 | 80.4% |

Note:
¹Based on loss on drying data.

Table 8 summarizes the yields at each step and the overall yields for the specific examples described above.

TABLE 8
Yield (%) of Each Step and Overall Yield

| Ex. | Step 1¹ | Step 2¹ | Step 3 | Overall yield (%) |
|---|---|---|---|---|
| 1 | 60.7 | 69.8 | 87 | 36.7 |
| 2 | 69.2 | 93.5 | 84.9 | 54.93 |
| 3 | 64.3 | 92.6 | 80.5 | 47.93 |
| 4 | 65.2 | N/A | N/A | N/A |
| 5 | 59.3 | 95.6 | 80 | 45.35 |
| 6 | 63.2 | 94.1 | 77 | 45.79 |
| 7 | 76.6 | 64.7 | N/A | 49.56 |
| 8 | 62.06 | N/A | N/A | N/A |

Note:
¹Based on loss on drying data.

What is claimed is:

1. A process for producing gabapentin comprising reacting gabapentin lactam with aqueous hydrobromic acid to yield gabapentin hydrobromide; and
neutralizing said gabapentin hydrobromide in a solution with at least one base to yield gabapentin.

2. The process of claim 1, further comprising the step of purifying said gabapentin.

3. The process of claim 2, wherein said step of purifying comprises at least one recrystallization process.

4. The process of claim 3, wherein said at least one recrystallization process comprises recrystallizing said gabapentin using at least one of methanol, isopropyl alcohol, water, aqueous methanol, aqueous isopropyl alcohol and combinations thereof.

5. The process of claim 1, wherein said step of reacting gabapentin lactam with aqueous hydrobromic acid comprises refluxing gabapentin lactam with aqueous hydrobromic acid.

6. The process of claim 1, wherein said aqueous hydrobromic acid is prepared by combining approximately one part of water for approximately every two parts of hydrobromic acid.

7. The process of claim 1, wherein said step of reacting gabapentin lactam with aqueous hydrobromic acid comprises reacting gabapentin lactam with aqueous hydrobromic acid in a ratio of approximately 1 kilogram of gabapentin lactam to approximately 1 liter of water to approximately 1 liter of hydrobromic acid.

8. The process of claim 1, further comprising the step of isolating said gabapentin hydrobromide.

9. The process of claim 8, wherein said step of isolating gabapentin hydrobromide is by at least one filtration process.

10. The process of claim 8, wherein said step of isolating gabapentin hydrobromide is by at least one centrifugation process.

11. The process of claim 1, further comprising the step of washing said gabapentin hydrobromide with at least one solvent.

12. The process of claim 11, wherein said at least one solvent is a ketonic solvent.

13. The process of claim 11, wherein said at least one solvent is at least one of methylethylketone, isopropyl alcohol and combinations thereof.

14. The process of claim 13, wherein said at least one solvent is methylethylketone.

15. The process of claim 9, further comprising the step of preserving at least one filtrate material from said at least one filtration process and obtaining at least one additional yield of gabapentin hydrobromide from said at least one filtrate material.

16. The process of claim 1, wherein said at least one base used in said step of neutralizing gabapentin hydrobromide in a solution to yield gabapentin is at least one organic base.

17. The process of claim 16, wherein said at least one organic base is at least one amine.

18. The process of claim 17, wherein said amine is at least one of tributylamine, diethyl amine, triethylamine, diisopropylamine, diisopropylethylamine, dibutylamine, di-(2-ethylhexylamine), dicyclohexylamine and combinations thereof.

19. The process of claim 1, wherein said step of neutralizing gabapentin hydrobromide in a solution with at least one base to yield gabapentin comprises adjusting the pH of said solution to a value between approximately 6.4 and 9.4.

20. The process of claim 19, wherein said pH is a value between approximately 7.2±0.8.

21. The process of claim 1, wherein said solution comprises gabapentin hydrobromide, at least one base and at least one of acetone, methanol, isopropyl alcohol, water, aqueous methanol, aqueous isopropyl alcohol and combinations thereof.

22. The process of claim 1, wherein said step of neutralizing gabapentin hydrobromide in a solution with at least one base to yield gabapentin comprises neutralizing with at least one ion exchange resin material.

23. The process of claim 1, further comprising the step of conducting at least one analytical test on at least one of said gabapentin hydrobromide and said gabapentin.

24. The process of claim 23, wherein said at least one analytical test includes measuring bromide content.

25. The process of claim 1 further comprising the step of seeding with at least one of gabapentin hydrobromide and gabapentin.
